(19) United States
(12) Patent Application Publication
(10) Pub. No.: US 2026/0048132 A1
(43) Pub. Date: Feb. 19, 2026

(54) IMIDAZOLYL GOLD COMPOUNDS FOR THE TREATMENT OF LUNG CANCER

(71) Applicant: University of Cincinnati, Cincinnati, OH (US)

(72) Inventors: Pier Paolo Scaglioni, Cincinnati, OH (US); Cristina Andreani, Cincinnati, OH (US); Caterina Bartolacci, Cincinnati, OH (US); Rossana Galassi, Camerino (IT); Stefania Pucciarelli, Camerino (IT)

(21) Appl. No.: 19/304,094

(22) Filed: Aug. 19, 2025

Related U.S. Application Data
(60) Provisional application No. 63/684,646, filed on Aug. 19, 2024.

(51) Int. Cl. A61K 47/54; A61P 35/00; C07F 1/00

(52) U.S. Cl. CPC A61K 47/547; A61P 35/00; C07F 1/00

(57) ABSTRACT

Provided herein are methods composition for treating lung cancer in a subject in need thereof, the method comprising administering to the subject an effective amount of an imidazolate gold compound according to Formula I, or a pharmaceutically acceptable salt, racemate, or enantiomer thereof, wherein Formula I is a gold complex having a structure Au(L)(L′)ₙ, wherein: n is an integer from 1 to 3; L is an imidazolyl-based N-heterocyclic carbene (NHC) ligand; and each L′ is independently selected from an imidazolyl-based NHC ligand, a triaryl phosphine, or a halide.

### CROSS-REFERENCE TO RELATED APPLICATIONS

This application claims priority to U.S. Provisional Application Ser. No. 63/684,646, filed Aug. 19, 2024, the entire contents of which are incorporated herein by reference.

### STATEMENT REGARDING FEDERALLY SPONSORED RESEARCH

This invention was made with government support under CA259845 awarded by National Institutes of Health. The government has certain rights in the invention

### TECHNICAL FIELD

This disclosure relates to methods of treating lung cancer by administering imidazolyl gold compounds.

### BACKGROUND

Approximately 80-85% of lung cancers are non-small cell lung cancer (NSCLC). The most common subtypes of NSCLC include adenocarcinoma, squamous cell carcinoma, and large cell carcinoma, while less common subtypes include adenosquamous carcinoma and sarcomatoid carcinoma. Although NSCLC typically progresses more slowly than small cell lung cancer (SCLC), 40% of NSCLCs have metastasized beyond the lungs by the time the cancer is diagnosed.

Despite advances in targeted therapies such as EGFR or KRAS inhibitors for certain genetic subsets of lung cancer, many patients, such as those with advanced, unresectable NSCLC lacking targetable mutations still rely on conventional treatments including radiotherapy and platinum-based chemotherapy. These standard regimens are often accompanied by serious side effects and limited efficacy.

Accordingly, a need exists to develop targeted anti-cancer therapies for treating NSCLC and minimizing side effects.

### SUMMARY

Accordingly, the present disclosure is directed to targeted therapies for lung cancer, namely, imidazolyl gold compounds.

In an embodiment, the present disclosure relates to a method for treating lung cancer in a subject in need thereof, the method comprising administering to the subject an effective amount of an imidazolate gold compound according to Formula I, or a pharmaceutically acceptable salt, racemate, or enantiomer thereof, wherein Formula I is a gold complex having a structure Au(L)(L′)ₙ, wherein: n is an integer from 1 to 3; L is an imidazolyl-based N-heterocyclic carbene (NHC) ligand; and each L′ is independently selected from an imidazolyl-based NHC ligand, a triaryl phosphine, or a halide.

These and other objects, features, embodiments, and advantages will become apparent to those of ordinary skill in the art from a reading of the following detailed description and the appended claims.

### BRIEF DESCRIPTION OF THE DRAWINGS

The details of embodiments of the presently-disclosed subject matter are set forth in this document. Modifications to embodiments described in this document, and other embodiments, will be evident to those of ordinary skill in the art after a study of the information provided herein.

FIG. 1 is illustrative molecular structures of representative gold complexes of the invention. This figure depicts the skeletal formulas of compounds 1-8 as described herein. Each structure shows the gold center (Au) coordinated by an N-heterocyclic ligand and additional co-ligands.

FIG. 2A depicts the in vitro cytotoxic activity of compound 1 against human NSCLC cell lines.

FIG. 2B depicts the in vitro cytotoxic activity of compound 2 against human NSCLC cell lines.

FIG. 2C depicts the in vitro cytotoxic activity of compound 3 against human NSCLC cell lines.

FIG. 2D depicts the in vitro cytotoxic activity of compound 4 against human NSCLC cell lines.

FIG. 2E depicts the in vitro cytotoxic activity of compound 5 against human NSCLC cell lines.

FIG. 2F depicts the in vitro cytotoxic activity of compound 6 against human NSCLC cell lines.

FIG. 2G depicts the in vitro cytotoxic activity of compound 7 against human NSCLC cell lines.

FIG. 2H depicts the in vitro cytotoxic activity of compound 8 against human NSCLC cell lines.

FIG. 3A depicts absolute Thioredoxin reductase (TrxR) activity normalized by the total milligrams of protein lysates in the perturbation of thioredoxin reductase activity by the gold compounds in NSCLC cell lines.

FIG. 3B depicts the mean difference TrxR activity (%) relative to the vehicle (veh) of each time point in the perturbation of thioredoxin reductase activity by the gold compounds in NSCLC cell lines.

FIG. 4 depicts the correlation between TrxR inhibition and cytotoxic potency of the gold complexes in H522 cells (left panel) and A549 cells (right panel).

FIG. 5A depicts the cell-free hDHFR catalyzed reaction velocity is differently inhibited by compound 7.

FIG. 5B depicts the cell-free hDHFR catalyzed reaction velocity is differently inhibited by compound 8.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to achieve the disclosed subject matter.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of”

As used herein the singular forms “a,” “an” and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

When the term “independently selected” is used, the substituents being referred to (e.g., R groups, such as groups R₁ and R₂), can be identical or different. For example, both R₁ and R₂ can be the same substituent, or R₁ and R₂ can each be different substituents selected from a specified group.

It should be understood that any two quantitative values assigned to a property may constitute a range of that property, and all combinations of ranges formed from all stated quantitative values of a given property are contemplated in this disclosure.

The term “subject” and “patient” as used herein may be used interchangeably and refer to an organism to be treated by the methods and compositions of the present disclosure. In some embodiments, such organisms are a mammal, optionally a human, mouse, rat, guinea pig, dog, cat, horse, cow, pig, or non-human primate, such as a monkey, chimpanzee, baboon, and rhesus. In some embodiments, the subject is a human.

The terms “treat,” “treatment,” and “treating,” and grammatical equivalents thereof as used herein, includes any effect of alleviating or abrogating a disease, disorder, and/or symptoms thereof, for example, lessening, reducing, modulating, ameliorating or eliminating, that results in the improvement of the condition, disease, disorder, and the like, or ameliorating a symptom thereof. Treating may include curing, improving, or at least partially ameliorating the disorder. In certain embodiments, treating is curing the disease.

The terms “pharmaceutically acceptable” and “pharmacologically acceptable,” as used herein, refer to compounds, molecular entities, compositions, materials, and/or dosage forms that do not produce an adverse, allergic or other untoward reaction when administered to an animal, or a human, as appropriate. For human administration, preparations generally meet sterility, pyrogenicity, and general safety and purity standards as required by FDA Office of Biologics standards. “Pharmaceutically acceptable” and “pharmacologically acceptable” may mean approved or approvable by a regulatory agency of the federal or a state government or the corresponding agency in countries other than the United States, or that is listed in the U.S. Pharmacopoeia or other generally recognized pharmacopoeia for use in animals.

As used herein, the terms “pharmaceutically acceptable excipient” or “pharmaceutically acceptable carrier” refer to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions, alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For additional examples of excipients, see e.g., Martin, Remington's Pharmaceutical Sciences, 15th Ed., Mack Publ. Co., Easton, Pa. (1975).

Δn “effective amount,” as used herein, refers to an amount of a substance (e.g., a therapeutic compound and/or composition) that elicits a desired biological response. In some embodiments, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some embodiments, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

As used herein the term “cancer” refers to any disease that results from the uncontrolled division of cells capable of metastasizing. The term “malignant tumor cell” and “cancer cell” may be used interchangeably throughout the specification.

Cancers that can be treated with compounds of the present invention include, but are not limited to: breast cancer including male breast cancer; digestive/gastrointestinal cancers including anal cancer, appendix cancer, extrahepatic bile duct cancer, gastrointestinal carcinoid tumor, colon cancer, esophageal cancer, gallbladder cancer, gastric cancer, gastrointestinal stromal tumors (“gist”), Islet cell tumors, adult primary liver cancer, childhood liver cancer, pancreatic cancer, rectal cancer, small intestine cancer, and stomach (gastric) cancer; endocrine and neuroendocrine cancers including pancreatic adenocarcinoma, adrenocortical carcinoma, pancreatic neuroendocrine tumors, Merkel cell carcinoma, non-small cell lung neuroendocrine tumor, small cell lung neuroendocrine tumor, parathyroid cancer, pheochromocytoma, pituitary tumor and thyroid cancer; eye cancers including intraocular melanoma and retinoblastoma; genitourinary cancer including bladder cancer, kidney (renal cell) cancer, penile cancer, prostate cancer, transitional cell renal pelvis and ureter cancer, testicular cancer, urethral cancer and Wilms tumor; germ cell cancers including childhood central nervous system cancer, childhood extracranial germ cell tumor, extragonadal germ cell tumor, ovarian germ cell tumor and testicular cancer; gynecologic cancers including cervical cancer, endometrial cancer, gestational trophoblastic tumor, ovarian epithelial cancer, ovarian germ cell tumor, uterine sarcoma, vaginal cancer and vulvar cancer; head and neck cancers including hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, parathyroid cancer, pharyngeal cancer, salivary gland cancer and throat cancer; leukemias including adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; lymphomas including AIDS-related lymphoma, cutaneous t-cell lymphoma, adult Hodgkin lymphoma, childhood Hodgkin lymphoma, Hodgkin lymphoma during pregnancy, mycosis fungoides, adult non-Hodgkin lymphoma, childhood non-Hodgkin lymphoma, non-Hodgkin lymphoma during pregnancy, primary central nervous system lymphoma, Sézary syndrome and Waldenström macroglobulinemia; musculoskeletal cancers including Ewing sarcoma, osteosarcoma and malignant fibrous histocytoma of bone, childhood rhabdomyosarcoma and soft-tissue sarcoma; neurological cancers including adult brain tumor, childhood brain tumor, astrocytomas, brain stem glioma, central nervous system atypical teratoid/rhabdoid tumor, central nervous system embryonal tumors, craniopharyngioma, ependymoma, neuroblastoma, primary central nervous system (CNS) lymphoma; respiratory/thoracic cancers including non-small cell lung cancer, small cell lung cancer, malignant mesothelioma, thymoma and thymic carcinoma; and skin cancers including Kaposi sarcoma, melanoma and squamous cell carcinoma. In some embodiments, the cancer is lung cancer, optionally non-small lung cancer (NSCLC). In some embodiments, the non-small cell lung cancer is a non-squamous NSCLC. In some embodiments, the NSLSC is a KRAS wild type tumor.

The term “N-heterocyclic carbene (NHC) ligand” as used herein refers to a neutral ligand containing a divalent carbon atom (carbene carbon) within a heterocyclic ring (typically a 5-membered ring with two nitrogens, such as an imidazol-2-ylidene). As used herein, NHC ligands are exemplified by, but not limited to, 1,3-disubstituted imidazol-2-ylidenes (e.g., 1,3-dimethyl-imidazol-2-ylidene or 1-benzyl-3-methyl-imidazol-2-ylidene) as well as related anionic imidazolyl ligands (obtained by deprotonation of an imidazole at an annular nitrogen). These ligands coordinate to gold through the carbene carbon or, in the anionic form, may bind through a carbon or nitrogen with a negative charge. The term “imidazolyl” as used herein encompasses both the neutral carbene form and the anionic form, unless otherwise specified.

The term “phosphine ligand” as used herein refers to a triarylphosphine coordinated to gold. Exemplary phosphines include, but are not limited to triphenylphosphine, tris(o-tolyl)phosphine, and TPPTS (trisodium 3,3′,3″-phosphanetriyltri(benzene-1-sulfonate)). In some embodiments, the phosphine ligand is triphenylphosphine (P(C₆H₅)₃, abbreviated as PPh₃).

The term “gold (I) complex” or “Au (I) complex” as used herein refers to a complex wherein gold is in the +1 oxidation state, typically two-coordinate (linear) or three-coordinate (T-shaped/trigonal) with a 14-16 electron configuration.

The term “gold (III) complex” or “Au (III) complex” as used herein refers to a complex where gold is in the +3 oxidation state, often four-coordinate (square planar, 16 electron) with three anionic ligands to balance charge.

The terms “halo,” “halide,” or “halogen,” as used herein, refer to fluoro (F), chloro (Cl), bromo (Br), and iodo (I) groups.

The terms “cyano” or “nitrile,” as used herein, refer to a —C—N functional group.

The term “alkyl,” as used herein, refers to a straight or branched saturated aliphatic hydrocarbon group having a single radical and 1-12 carbon atoms (i.e., C1-C12 alkyl). Non-limiting examples of alkyl groups include methyl, propyl, isopropyl, butyl, n-butyl, isobutyl, sec-butyl, tert-butyl, pentyl, hexyl, heptyl, octyl, nonyl, and the like. A branched alkyl means that one or more alkyl groups such as methyl, ethyl, or propyl replace one or both hydrogens in a —CH₂-group of a linear alkyl chain. In certain embodiments, alkyl is a C1-C6 alkyl or a C1-C4 alkyl. In other embodiments, alkyl is selected from the group consisting of methyl, ethyl, propyl, isopropyl, butyl, n-butyl, isobutyl, sec-butyl, and tert-butyl.

Alkyl groups can optionally be unsubstituted or substituted (a “substituted alkyl”) with one or more alkyl group substituents, which can be the same or different. The term “alkyl group substituent” includes but is not limited to alkyl, substituted alkyl, halo, hydroxyl, carboxyl, oxo, and the like. There can be optionally inserted along the alkyl chain one or more oxygen, sulfur or substituted or unsubstituted nitrogen atoms, wherein the nitrogen substituent is hydrogen or alkyl.

The term “hydroxyl,” as used herein, refers to a —OH functional group.

The term “carboxyl,” as used herein, refers to a —COOH functional group.

The term “oxo,” as used herein, refers to a ═O functional group.

A “pharmaceutically acceptable salt” is a cationic salt formed at any acidic (e.g., hydroxamic or carboxylic acid) group, or an anionic salt formed at any basic (e.g., amino) group. Many such salts are known in the art, as described in WO 1987/005297, by Johnston et al., published Sep. 11, 1987. Specific cationic salts include the alkali metal salts (such as sodium and potassium), and alkaline earth metal salts (such as magnesium and calcium) and organic salts. Specific anionic salts include halide (such as chloride, bromide, or fluoride salts), sulfate, and maleate. In embodiments, suitable pharmaceutically acceptable salts include, but are not limited to, halide, sodium, sulfate, acetate, phosphate, diphosphate, potassium, maleate, calcium, citrate, mesylate, nitrate, tartrate, aluminum, gluconate, carboxylate, and the like.

Such salts are well understood by the skilled artisan and the skilled artisan is able to prepare any number of salts given the knowledge in the art. Furthermore, it is recognized that the skilled artisan may select one salt over another for reasons of solubility, stability, formulation ease and the like. Determination and optimization of such salts is within the purview of the skilled artisan's practice.

The terms “enantiomer” and “racemate” have the standard art recognized meanings (see, e.g., Hawley's Condensed Chemical Dictionary, 16th ed. (2016)). The illustration of specific protected forms and other derivatives of the compounds of the instant invention is not intended to be limiting. The application of other useful protecting groups, salt forms, esters, and the like is within the purview of the skilled artisan.

One target for lung cancer therapy is thioredoxin reductase (TrxR). Thioredoxin reductases are enzymes that catalyze the reduction of thioredoxin and hence are a central component in the thioredoxin system. Electrons are taken from NADPH via TrxR and are transferred to the active site of Trx, which goes on to reduce protein disulfides or other substrates. Since the activity of TrxR is essential for cell growth and survival, it is a good target for anti-tumor therapy.

Another important target in lung cancer therapy is dihydrofolate reductase (DHFR), an enzyme required for DNA precursor synthesis. Antifolate drugs (DHFR inhibitors such as methotrexate or pemetrexed) are currently used in certain lung cancer treatment protocols. However, to date there have been few studies on gold compounds inhibiting human DHFR. For example, and without being bound by theory, a gold-based drug capable of dual targeting concurrently inhibiting TrxR and DHFR may provide a therapeutic modality against NSCLC, potentially overcoming resistance and reducing the likelihood of cancer cell survival.

Accordingly, the present disclosure addresses these gaps by providing a series of structurally related gold complexes with N-heterocyclic carbene or imidazolyl ligands that target both TrxR and DHFR for the treatment of cancer.

### Imidazolyl Gold Compounds

In some embodiments, the present disclosure is directed to gold complexes comprising N-heterocyclic carbene (NHC) or imidazolyl ligands in combination with phosphine and/or halide ligands, having the general structures described herein. These complexes exploit dual biological activities for the treatment of cancer: inhibition of thioredoxin reductase (TrxR) and inhibition of dihydrofolate reductase (DHFR), thereby inducing cancer cell death through two independent but complementary pathways, as demonstrated in the examples.

In embodiments, imidazolate gold compounds, or pharmaceutically acceptable salts, racemates, or enantiomers thereof, have a structure according to Formula I, as described in greater detail herein. In some embodiments, gold is in the +3 state (Au (III)). In some embodiments, gold is in the +1 states (Au (I)).

In some embodiments the gold complex is an Au (I) complex. In certain embodiments, the compound is a gold(I) complex with a linear or trigonal coordination geometry around the gold center. In some embodiments, the gold complex is an Au (III) complex with the general formula (NHC)—AuX₃ where X is a halogen (Cl or Br). These complexes typically adopt a square-planar or distorted geometry around gold(III), with the NHC bound at one coordination site and three halo ligands completing the coordination sphere.

These Au (III) complexes may be obtained by oxidizing the corresponding Au (I) precursors (for instance, by halogen addition to a linear Au(I)-NHC-halide complex).

In some embodiments the complex is a mixed-ligand NHC and phosphine complex. In some embodiments, the Formula I compound is an (NHC)—Au-PPh3 complex where NHC denotes an N-heterocyclic carbene or related imidazolyl ligand and PPh₃ is triphenylphosphine.

In some embodiments, the Formula I compound is a linear Au (I) complex of formula (C₉H₆Cl₂N₂)Au(P(C₆H₅)₃), comprising a 1-benzyl-4,5-dichloroimidazolyl ligand bound to Au and a triphenylphosphine ligand. Optionally, this neutral complex has a C(Au)—Au—P coordination, where the imidazolyl carbon binds to Au (I) (formally as a carbanion ligand) and PPh₃ occupies the second coordination site.

The gold complexes of Formula I are generally gold complexes having the form Au(L)(L′)ₙ, wherein n is an integer from 1 to 3. L is generally an imidazolyl-based NHC ligand as described in greater detail herein. Each L′ is a ligand independently selected from an imidazolyl-based NHC ligand, a phosphine (optionally a triaryl phosphine, optionally triphenylphosphine (PPh₃)), or a halide. In some embodiments, L′ is a halide. Optionally, each halide is independently selected from fluorine, chlorine, bromine, or iodine. In some embodiments, each halide is independently selected from chlorine or bromine. In some embodiments, each halide is chlorine.

In some embodiments, each halide is bromine.

In some embodiments, the gold complex is chosen from a complex having one of the following structures: Au(L)X₃; Au(L)(PPh₃); Au(L)X; or Au(L)(PPh₃)X; Au(L)₂]⁺A⁻, where A⁻ is a halide or an AuX₂⁻.

In some embodiments, a gold complex or a pharmaceutically acceptable salt thereof has a formula of Au L₁L₂, wherein L₁ is an N-heterocyclic carbene or imidazolyl ligand bound through a carbon or nitrogen atom. In some embodiments, L₁ comprises a 1,3-disubstituted imidazole-2-ylidene or imidazol-2-ide ligand optionally substituted with one or more halo groups on the imidazole ring. In some embodiments, L₂ is a ligand selected from the group consisting of halide, triarylphosphine, and a second N-heterocyclic carbene or imidazolyl ligand, with the proviso that when L₂ is a second N-heterocyclic carbene, the complex is cationic and accompanied by a balancing anion.

In some embodiments, a gold complex or a pharmaceutically acceptable salt thereof has a formula of Au(L)X. wherein L is an N-heterocyclic carbene or imidazolyl ligand bound through a carbon or nitrogen atom. In some embodiments, L is selected from 1,3-dimethylimidazol-2-ylidene or 1-benzyl-3-methylimidazol-2-ylidene. In some embodiments, X is a chlorine or bromide

In some embodiments, a gold complex or a pharmaceutically acceptable salt thereof has a formula of Au(L)X₃. wherein L is an N-heterocyclic carbene or imidazolyl ligand bound through a carbon or nitrogen atom. In some embodiments, L is selected from 1,3-dimethylimidazol-2-ylidene or 1-benzyl-3-methylimidazol-2-ylidene. In some embodiments, X₃ is three chlorines, three bromines, or a combination thereof.

In some embodiments, the phosphine ligand is a triarylphosphine, optionally triphenylphosphine, though other triaryl or tri(alkyl) phosphines are contemplated and possible.

In some embodiments, the gold complex is a trigonal Au (I) complex of formula (C₅H₈N₂)Au(P(C₆H₅)₃)Cl, featuring a 1,3-dimethylimidazol-2-ylidene (NHC) ligand, a triphenylphosphine, and a chlorido ligand coordinated to gold.

It will be appreciated that variations in the ligand substituents that maintain the core characteristics (presence of an NHC or imidazolyl ligand and either halide and/or phosphine co-ligands) are within the scope of this disclosure. For instance, imidazolyl ligands with different ring substituents (e.g., other electron-withdrawing groups instead of Cl, such as CF₃ or other halogens; or different N1/N3 alkyl groups) may be employed to tune the properties of the complexes. Likewise, while triphenylphosphine is primarily exemplified, other phosphines (e.g., tri-p-tolylphosphine or water-soluble phosphines for specialized uses) can coordinate to gold in a similar fashion.

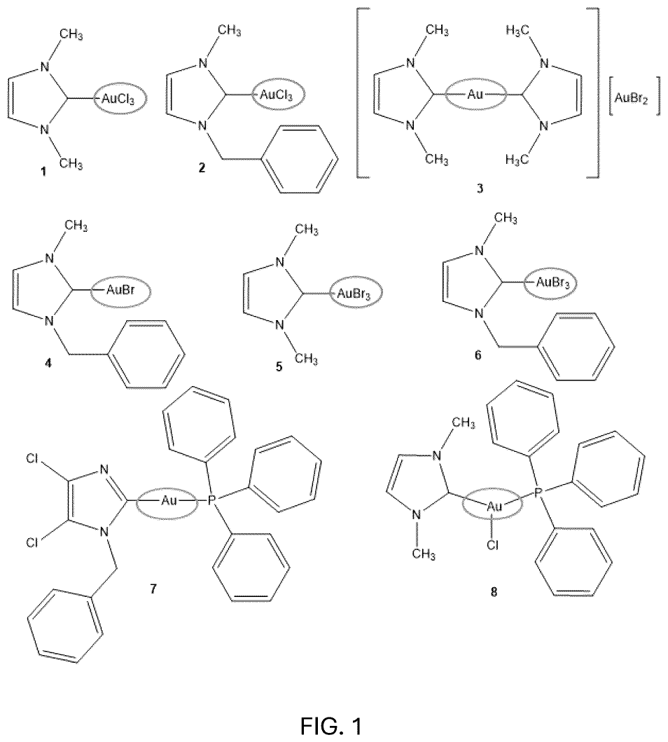

In some embodiments, the gold complex is selected from the compounds of FIG. 1, also depicted in Table 1, and described in greater detail herein.

### Imidazolyl-Based NHC Ligands

In some embodiments, the imidazolyl-based N-heterocyclic (NHC) ligand is a substituted imidazol-2-ylidene of the form C₃N₂(R¹)(R²)(R³)(R⁴), where R¹ and R² are substituents on the ring nitrogens selected from C₁-C₆ alkyl or benzyl, and R³ and R⁴ are substituents at the 4- and 5-positions of the imidazole ring selected from H, halogen, or C₁-C₂ alkyl.

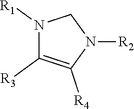

In some embodiments, R³ and R⁴ are each independently H or Cl. Optionally, R³ and R⁴ are both Cl. In some embodiments R³ and R⁴ are both Cl. In some embodiments, R¹ is benzyl. In some embodiments, R¹ is methyl. In some embodiments, R² is methyl.

In some embodiments, the imidazolyl-based N-heterocyclic (NHC) ligand is selected from 1-benzyl-3-methyl-4,5-dichloroimidazol-2-yl; 1,3-dimethylimidazol-2-ylidene; or 1-benzyl-4,5-dichloroimidazol-2-ide.

In some embodiments, the ligand L in formula (I) is a 1,3-disubstituted imidazol-2-ylidene or a 1-benzyl-substituted imidazolyl. In some embodiments, the benzyl substituent may carry other ring substituents such as halo. Suitable R¹ and R² substituents on the imidazole ring nitrogens include, for example, C₁-C₄ alkyl (methyl, ethyl, iso-propyl, etc.), benzyl, or other arylalkyl up to C₈. One substituent may be aromatic (benzyl or phenyl) while the other is alkyl, as exemplified by benzyl/methyl.

In some embodiments, R¹ is benzyl and R² is methyl. In some embodiments, R¹ and R² are both methyl. The ring carbon atoms at the 4- and 5-positions of the imidazole may independently be H or substituted (e.g., R³ and R⁴ can be H or halogen). In some embodiments, R³ and R⁴ are both halo, optionally chloro. When R³ and R⁴ are H, the imidazole is unsubstituted at those positions.

In some embodiments, the NHC ligand is 1-benzyl-3-methyl-4,5-dichloroimidazol-2-ylidene, which coordinates as an anionic imidazolyl ligand in the gold(I) complex. Another preferred ligand is 1,3-dimethylimidazol-2-ylidene.

In some embodiments, the gold complex is a linear gold (I) complex of formula Au(L)X or a pharmaceutically acceptable salt thereof, where L is 1,3-dimethylimidazol-2-ylidene and X is chloride or bromide.

In some embodiments, the gold complex is a linear gold complex of formula Au(L)(PPh₃) or a pharmaceutically acceptable salt thereof. Optionally, L is 1-benzyl-4,5-dichloroimidazol-2-ide.

In some embodiments, the gold complex is a trigonal gold(I) complex of formula Au(L)(PPh₃)X or a pharmaceutically acceptable salt thereof, where L is 1,3-dimethylimidazol-2-ylidene and X=Cl).

In some embodiments, the gold complex is a bis(NHC) cationic gold(I) complex [Au(NHC)₂]⁺A⁻, where A⁻ is a halide or an AuX₂ anion (e.g., [Au(1,3-dimethylimidazol-2-ylidene)₂]⁺[AuBr₂]⁻.

In some embodiments, the gold complex is a gold complex of formula Au(L)X₃ or a pharmaceutically acceptable salt thereof, wherein L is an NHC ligand as described herein, and X is a halogen selected from chloride or bromide.

In some embodiments, the gold complex is selected from 1,3-dimethylimidazol-2-ylidene)gold (III) trichloride (compound 1), (1-benzyl-3-methylimidazol-2-ylidene)gold (III) trichloride (compound 2), (1,3-dimethylimidazol-2-ylidene)gold(III) tribromide (compound 5), or (1-benzyl-3-methylimidazol-2-ylidene)gold(III) tribromide (compound 6).

Suitable Formula I compounds for use in the methods disclosed herein include any of the compounds set forth in Table 1, alone or in combination.

TABLE 1
Formula I compounds

| | |
|---|---|
| Compound 1 | 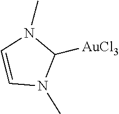 |
| Compound 2 | 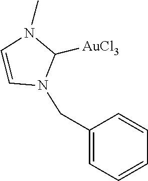 |
| Compound 3 | 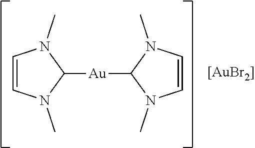 |
| Compound 4 | 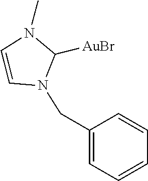 |
| Compound 5 | 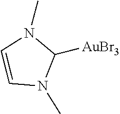 |
| Compound 6 | 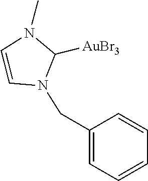 |
| Compound 7 | 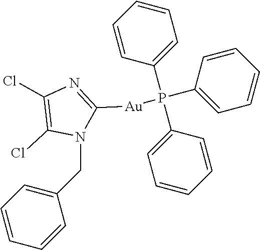 |
| Compound 8 | 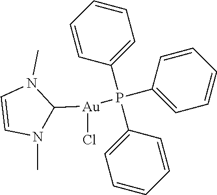 |

### Synthesis and Characterization

The gold complexes of the present disclosure may be synthesized via a series of logical steps from readily available starting materials, using techniques of organometallic and coordination chemistry. General methods for preparing such complexes include: (a) carbene transfer or ligand substitution reactions to introduce NHC or phosphine ligands to gold(I), (b) halide metathesis and counterion exchange to form cationic or anionic complexes, and (c) oxidative addition of halogens to gold(I) precursors to yield gold(III) complexes. The following synthetic routes and examples illustrate the preparation of the exemplary compounds.

### Synthesis of Gold(III) NHC Trihalide Complexes (Compounds 1 and 2):

In some embodiments a gold(III) trichloride NHC complex may be obtained by oxidizing a gold(I)-NHC-halide precursor with a halogenating agent. For example, to prepare compound 1, (1,3-dimethylimidazol-2-ylidene)Au(I)-Cl (an initial linear Au(I) complex) is treated with an oxidant such as iodobenzene dichloride (PhICl₂) in a suitable solvent (e.g., acetonitrile). This causes oxidative addition of Cl₂ to the gold center, yielding the gold(III) dichloride adduct, which rearranges to the trichloride complex Au(NHC)(Cl)₃. The product can be isolated as a yellow solid in high yield (e.g., ˜98% yield for compound 1). Characterization of compound 1 by elemental analysis (found values consistent with C₅H₈N₂AuCl₃) and mass spectrometry confirms the formulation.

Similarly, compound 2 (1-benzyl-3-methylimidazol-2-ylidene AuCl₃) is prepared by reacting (1-benzyl-3-methylimidazol-2-ylidene)AuCl with PhICl₂ under the same conditions. After stirring and workup, compound 2 is obtained as a yellow solid (ca. 88% yield), and its analytical data (C₁₁H₁₂N₂AuCl₃ by EA, and diagnostic peaks in ESI-MS for the bis-NHC Au cation and related fragments) verify its identity. These methods demonstrate a straightforward one-pot conversion of Au(I)-NHC to Au(III)-NHC complexes using halogen transfer reagents.

### Halide Exchange and Formation of Bis(NHC) Cationic Complexes (Compounds 3 and 4):

Gold(I) complexes with different halides or multiple NHC ligands may be prepared by metathesis. Compound 3 was synthesized by treating a chloro-gold(I) NHC with a bromide source to induce halide exchange and dimerization. In practice, dissolving (1,3-dimethylimidazol-2-ylidene)AuCl in acetone and adding excess sodium bromide causes the replacement of the chloride ligand with bromide. Under these conditions, a portion of the Au(I) complexes undergo halide bridging to form a dinuclear complex where one Au(I) is coordinated by two NHC ligands (becoming a cation) and the other Au retains two bromides as an anionic counterion. The product, isolated as a white solid after filtration and solvent removal, is the bis-NHC gold(I) bromidoaurate, [Au(NHC)₂]⁺[AuBr₂]⁻ (compound 3), obtained in 74% yield. Its elemental analysis corresponds to C₁₀H₁₆N₄Au₂Br₂ (consistent with two gold atoms per formula unit, each paired cation/anion). ESI-MS further confirms the presence of the bis-carbene cation (m/z 389 for [(NHC)_2Au]⁺) and the AuBr₂ anion (m/z 356 in negative mode).

On the other hand, compound 4 (a neutral monocarbene Au(I) bromide) may be obtained when a similar procedure is applied to the benzyl-substituted analog: (1-benzyl-3-methylimidazol-2-ylidene)AuCl treated with NaBr yields (1-benzyl-3-methylimidazol-2-ylidene)AuBr as the major product. Compound 4 is isolated in ˜91% yield as a white solid. Elemental analysis (C₁₁H₁₂N₂AuBr) confirms the formula and the mass spectrum shows the characteristic [(NHC)_2Au]⁺ peak (m/z 541 for the benzyl/methyl NHC case) indicating some association in the ionization process, but the molecular composition corresponds to a 1:1 NHC:Au:Br complex. The solid-state structure of compounds 3 and 4 can be elucidated by X-ray crystallography as needed (indeed, prior studies have characterized analogous mono- and bis-NHC Au complexes). These examples illustrate that simple halide metathesis can yield either ionic or neutral Au(I) NHC complexes depending on the substituents and reaction stoichiometry; both types are within the scope of the disclosure.

### Oxidation to Gold (III) Tribromide Complexes (Compound 5 and 6):

By oxidizing the Au(I) bromide complexes described above, one can obtain the Au (III) tribromide analogs. For instance, compound 5 (1,3-dimethylimidazol-2-ylidene)AuBr₃ was prepared by treating the bis-NHC dibromidoaurate (compound 3) with elemental bromine (Br₂) in acetonitrile. Bromine addition promotes oxidation of Au (I) to Au (III) and incorporation of additional bromide. After a short reaction (e.g., 2 h at room temperature) and workup, compound 5 is isolated as a dark orange solid in ˜93% yield. Elemental analysis (C₅H₈N₂AuBr₃) confirms its composition.

Similarly, compound 6 (1-benzyl-3-methylimidazol-2-ylidene)AuBr₃ can be obtained by adding Br₂ to (1-benzyl-3-methylimidazol-2-ylidene)AuCl (or to the analogous AuBr, which can be generated in situ by halide exchange). The reaction yields an orange solid (84% yield) identified as the Au (III) tribromide complex. Elemental analysis (C₁₁H₁₂N₂AuBr₃) matches the expected formula. These synthetic pathways show that halo-oxidation is a versatile method to access Au (III) complexes from Au(I) precursors, allowing the preparation of both tri-chloro and tri-bromo complexes depending on the halogen used (PhICl₂ vs Br₂).

### Formation of Gold(I) Phosphine Complexes (Compounds 7 and 8):

Compound 7 was synthesized by a metalation and ligand substitution strategy. First, 1-benzyl-4,5-dichloroimidazole (the free N-heterocycle) is deprotonated with a strong base (e.g., n-butyllithium) at low temperature to generate the nucleophilic imidazolyl anion. In the same flask, a gold(I) precursor with a labile ligand is introduced—in this case, triphenylphosphine gold(I) hexafluorophosphate (PPh₃AuPF₆) is added to the THE solution containing the lithiated imidazolyl. The imidazolyl anion rapidly coordinates to the Au(PPh₃)⁺ fragment, displacing the PF₆⁻ counterion and yielding the neutral Au(imidazolyl)(PPh₃) product. After stirring (at 0° C. then room temperature) and standard aqueous workup to remove lithium salts and HF₆ byproducts, compound 7 is obtained as a brown crystalline solid. Recrystallization (e.g., from chloroform/hexane) affords pure compound 7 in moderate yield (the process yielded ˜40 mg from 0.6 mmol gold precursor, which is roughly 20-30% yield). Compound 7 is characterized by \hat{ }1H NMR (notably showing the benzyl CH₂ at δ˜5.3 and aromatic protons 7.2-7.5), \hat{ }31P NMR (a singlet at δ˜42 indicating the PPh₃ bound to Au(I)), and elemental analysis consistent with C₂₈H₂₂Cl₂N₂PAu. High-resolution ESI-MS in positive mode shows a prominent peak at m/z 684.6 corresponding to [Imidazolyl(Au)PPh₃]⁺ (where Imidazolyl=C₉H₆Cl₂N₂ fragment), along with the characteristic [(PPh₃)₂Au]⁺ peak at m/z 721. These data confirm that compound 7 is indeed the Au(I) complex with a dichlorinated imidazolyl ligand and a triphenylphosphine, with no halide directly bound (the Au—C and Au—P bonds completing the coordination).

Compound 8 was prepared by a direct ligand substitution on Au(I). In one convenient method, a chlorido gold(I) NHC complex is treated with triphenylphosphine, leading to displacement of a labile ligand (often forming a coordination polymer initially) and formation of the monomeric Au(NHC)(PPh₃)Cl complex. For example, (1,3-dimethylimidazol-2-ylidene)AuCl (50 mg) is dissolved in dichloromethane and combined with equimolar PPh₃ (approximately 1:1 ratio). The mixture is stirred at ambient temperature (e.g., 18 h) allowing PPh₃ to coordinate to gold, after which the solvent is removed and the residue washed with hexane to give compound 8 as a white microcrystalline solid. Yield is around 45% for this substitution reaction. Compound 8 is characterized by \hat{ }1H NMR (aromatic protons of PPh₃ around δ 7.5-7.6, the imidazolylidene ring CH at ˜7.2, and the N—CH₃ at ˜3.8-4.0 ppm), and by \hat{ }31P NMR (singlet at δ˜33 in CDCl₃). Elemental analysis confirms C₂₃H₂₃N₂ClP Au (accounting for one PPh₃, one C₅H₈N₂, and one Cl). ESI-MS shows a peak at m/z 554.9 for [Au(NHC-Me₂)(PPh₃)]⁺ and again the diphosphine Au fragment at m/z 720.9. These results are consistent with the formulation of compound 8 as (1,3-dimethylimidazol-2-ylidene)Au(PPh₃)Cl. Notably, the persistence of a distinct Au—Cl stretch in the IR spectrum of compound 8 (at ˜329 cm⁻¹, matching a reference PPh₃AuCl) was noted as evidence of the Au—Cl bond, confirming the trigonal structure assignment.

### Methods of Treating Lung Cancer

In some embodiments, a method of treating lung cancer in a subject in need thereof is provided, the method comprising administering to the subject an effective amount of an imidazolate gold compound according to Formula I, or a pharmaceutically acceptable salt, racemate, enantiomer, or derivative thereof. In another embodiment, a compound according to Formula I, or a pharmaceutically acceptable salt, racemate, enantiomer, or derivative thereof is provided, for use in treating lung cancer.

In some embodiments, the Formula I compound is selected from compounds 1-8. In some embodiments, the Formula I compound is compound 7. In some embodiments, the Formula I compound is compound 8. The disclosed gold complexes may be used as anticancer agents, especially against lung cancers such as NSCLC. In vitro studies, described in greater detail herein, demonstrate that these compounds induce potent cytotoxic effects in a panel of human NSCLC cell lines, including both KRAS-mutant and wild-type lines.

In some embodiments, the Formula I compound displays a favorable selectivity profile, exhibiting less toxicity toward normal (non-cancerous) cells relative to other cancer treatments. For example, as shown in the examples, compounds of the present disclosure tested against non-tumorigenic human lung fibroblasts (IMR-90) and showed significantly higher IC50 values in these normal cells, indicating a wide therapeutic window. This selectivity suggests that the disclosed compounds can kill cancer cells while sparing healthy cells, a highly desirable attribute for chemotherapy. Without being limited by theory, this selectivity may stem from cancer-specific uptake or the cancer cells' greater reliance on TrxR/DHFR pathways, making them more susceptible to the drugs' dual-action mechanism.

In some embodiments, the lung cancer is non-small cell lung cancer (NSCLC). In embodiments, the lung cancer is non-small cell lung cancer (NSCLC). In another embodiment, the lung cancer is KRAS-WT or KM lung cancer. In a specific embodiment, the lung cancer is KRAS-WT lung cancer.

Optionally, administration of the compound may be by intravenous injection, infusion, or another suitable route, and the effective amount is that which results in inhibition of tumor growth or reduction of tumor size. In some embodiments, the compositions provided herein may be used to treat NSCLC tumors that are resistant to platinum-based chemotherapy or that lack actionable mutations (e.g., KRAS wild-type tumors), by leveraging the gold complex's dual mechanism of action to induce cancer cell death.

In some embodiments, administering the compounds comprises dosing on an intermittent schedule (for example, 1-3 times per week dosing) to achieve sufficient tumor exposure while allowing normal tissue recovery, The method may be applied to primary lung tumors or metastatic lesions thereof, and can result in apoptosis and growth inhibition of the cancer cells through TrxR and DHFR pathway suppression. wherein the compound inhibits thioredoxin reductase and dihydrofolate reductase in cancer cells, thereby inducing oxidative stress and cell cycle arrest in said cancer cells.

It will be appreciated that the methods described herein may be carried out in vitro (e.g., in a research or diagnostic context) to study redox and folate metabolism pathways, or in vivo as part of a therapeutic mechanism.

In some embodiments, treatment with the gold complex causes at least about 20%, about 25%, about 30%, about 35%, about 40%, about 45%, about 50%, about 55%, about 60%, about 65%, about 70%, about 75%, about 80% or more reduction in TrxR activity in the cell, including any range defined by any two of the aforementioned endpoints.

In some embodiments, treatment with the gold complex causes at least about 20%, about 25%, about 30%, about 35%, about 40%, about 45%, about 50%, about 55%, about 60%, about 65%, about 70%, about 75%, about 80% or more inhibition of DHFR activity, including any range defined by any two of the aforementioned endpoints.

In embodiments, the Formula I compound is administered orally, intravenously, intraperitoneally, intrathecally, intramuscularly, subcutaneously, rectally, intravaginally, sublingually, via inhalation, or transdermally. In some embodiments, the Formula I compound is administered parenterally, e.g., by injection or infusion.

The treatment may further comprise co-administering an additional therapy such as an antifolate (e.g., pemetrexed) or an immune checkpoint inhibitor, in which case the gold complex can provide complementary activity (e.g., TrxR inhibition to increase oxidative stress in cancer cells, making them more susceptible to immune-mediated killing). The gold complex, when formulated into a medicament, is intended to reduce tumor cell viability by simultaneously targeting cellular redox regulation and folate metabolism pathways in the cancer.

In embodiments, the methods disclosed herein further comprise administering to the subject one or more additional anti-cancer agents. Suitable anti-cancer agents include one or more agents selected from a chemotherapeutic agent, an immunotherapeutic agent, and radiation therapy.

In some embodiments, the additional chemotherapeutic agent is selected from the group consisting of cisplatin, carboplatin, paclitaxel, albumin-bound paclitaxel, docetaxel, gemcitabine, vinorelbine, etopside, pemetrexed, and combinations thereof.

In some embodiments, the additional immunotherapeutic agent is selected from the group consisting of nivolumab, pembrolizumab, cemiplimab, atezolizumab, durvalumab, ipilimumab, tremelimumab, and combinations thereof.

In embodiments, the Formula I compound and the one or more additional anti-cancer agents are co-administered. “Co-administered,” as used herein, refers to administration of the Formula I compound and the additional anti-cancer agent such that both agents can simultaneously achieve a physiological effect, e.g., in a recipient subject. The two agents, however, need not be administered together. In certain embodiments, administration of one agent can precede administration of the other. Simultaneous physiological effect need not necessarily require presence of both agents in the circulation at the same time. However, in certain embodiments, co-administering results in both agents being simultaneously present in the subject. Thus, in embodiments, the Formula I compound and the additional anti-cancer agent may be administered concurrently or sequentially.

### Pharmaceutical Compositions

In embodiments, a pharmaceutical composition is provided, the composition comprising a compound according to Formula I, or a pharmaceutically acceptable salt, racemate, enantiomer, or derivative thereof; and at least one pharmaceutically acceptable carrier. In embodiments, the pharmaceutical compositions disclosed herein are formulated for the treatment of lung cancer, i.e., for administration to a patient suffering from lung cancer.

The pharmaceutically acceptable excipient, or carrier, must be “acceptable” in the sense of being compatible with the other ingredients of the composition and not deleterious to the recipients thereof. The disclosure further includes a pharmaceutical composition, in combination with packaging material suitable for the pharmaceutical composition, including instructions for the use of the composition in the treatment of subjects in need thereof.

Pharmaceutical compositions include those suitable for oral, intravenous, intraperitoneal, intrathecal, intramuscular, subcutaneous, rectal, vaginal, sublingual, inhalation, or transdermal administration. In a specific embodiment, the pharmaceutical compositions are formulated for intravenous administration, e.g., by injection or infusion.

The compositions may be prepared by any methods well known in the art of pharmacy, for example, using methods such as those described in Remington: The Science and Practice of Pharmacy (21st ed., Lippincott Williams and Wilkins, 2005, see Part 5: Pharmaceutical Manufacturing). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given vaccine composition.

For parenteral administration, suitable compositions include aqueous and non-aqueous sterile suspensions for intravenous administration or injection administration. The compositions may be presented in unit dose or multi-dose containers, for example, sealed vials and ampoules.

As will be understood by those of skill in this art, the specific dose level for any particular subject will depend on a variety of factors, including the activity of the agent employed; the age, body weight, general health, and sex of the individual being treated; the time and route of administration; the rate of excretion; and the like.

In embodiments, an effective dose of a Formula I compound according to the present disclosure may range from about 0.01 mg/kg/day to about 100 mg/kg/day, or from about 0.01 mg/kg/day to about 10 mg/kg/day, or from about 0.1 mg/kg/day to about 100 mg/kg/day, or from about 0.1 mg/kg/day to about 10 mg/kg/day, or from about 1 mg/kg/day to about 10 mg/kg/day. In embodiments, the dose of a Formula 1 compound is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 30, 40, 50, 60, 70, 80, 90, or 100 mg/kg/day, or any selected range of values therebetween.

### Mechanistic Insights

The dual inhibition of TrxR and DHFR by the disclosed complexes provides a mechanistic rationale for their potent anticancer effects. TrxR is a selenocysteine-containing enzyme that gold compounds readily inhibit by binding to the active site selenol/thiol, leading to enzyme inactivation. All tested complexes showed some degree of TrxR inhibition in NSCLC cells. Without being bound by theory, it is hypothesized that the phosphine ligand increases the lipophilicity of the complex, thereby aiding membrane permeability, and also stabilizes the gold center against premature reduction or ligand loss, allowing more gold to reach intracellular targets (TrxR in the cytosol).

The allosteric inhibition of the present compounds suggest binding to a regulatory or less-conserved site on DHFR, possibly at the dimer interface or a pocket distinct from the active site, which becomes more influential at higher substrate (dihydrofolate) concentrations, leading to reduced catalytic turnover. This is unusual because DHFR is a monomeric enzyme in humans (unlike bacterial DHFR which can have feedback loops), yet the complexes of the present disclosure induced cooperative inhibition kinetics.

Another mechanistic aspect is the selectivity for cancer cells. Cancer cells, especially those with oncogenic KRAS, often have upregulated antioxidant systems (TrxR/Trx, glutathione) and higher folate metabolism demands. The disclosed compounds are believed to exploit these vulnerabilities: KRAS-wild type lines (which rely heavily on folate cycle for growth) were very sensitive to compounds 3 and 4 (C—Au—Br environment), whereas KRAS-mutant lines (with high basal ROS) were broadly sensitive to the phosphine-containing compounds 7 and 8. This suggests that tumors without KRAS mutations (which cannot be treated with KRAS inhibitors) might particularly benefit from these gold complexes as they show effectiveness irrespective of KRAS status and can complement current therapies. Moreover, combining a TrxR inhibitor with an antifolate in one molecule could mimic a combination therapy (e.g., a platinum drug+pemetrexed regimen, which is standard for many NSCLC) but potentially with a single agent, thereby reducing side effects.

### EXAMPLES

The following examples are given by way of illustration and are in no way intended to limit the scope of the present disclosure.

### Example 1: Cytotoxicity and Enzyme Inhibition Assays

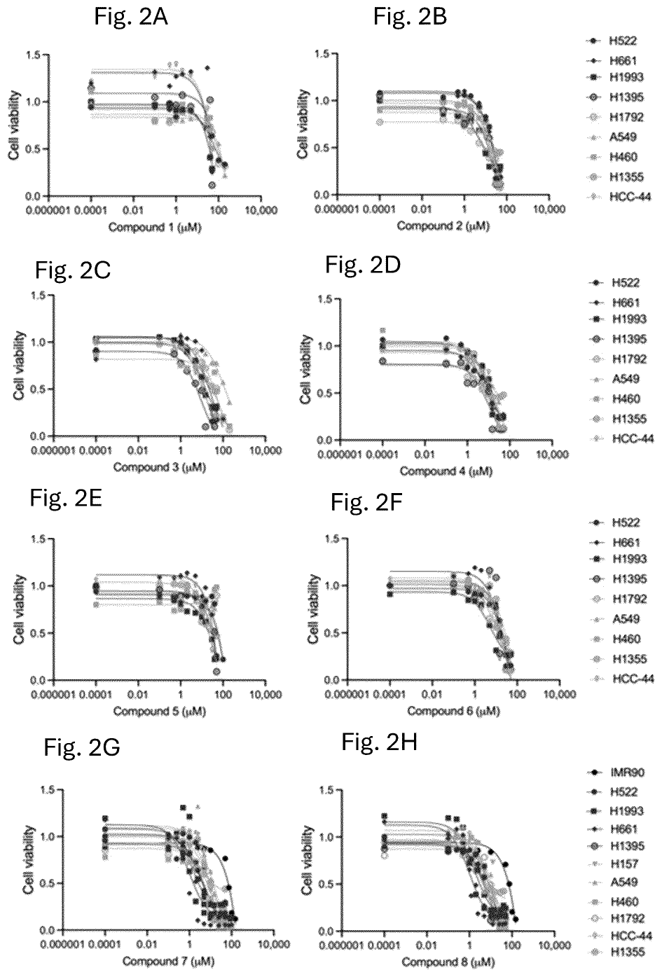

Each of compound 1-8 were evaluated for anticancer activity against a panel of human NSCLC cell lines, including H522, H1395, HCC-44 (KRAS wild-type) and A549, H157, H460 (KRAS mutant), among others. Cells were treated with compounds over a range of concentrations for 48 h, and viability was assessed by MTT assay (measuring mitochondrial metabolic activity). IC50 values were determined from dose-response curves, as shown in FIG. 2 and are summarized in Table 2.

TABLE 2

| # | H522 | H661 | H1395 | H1993 | IC₅₀ * | H157 | A549 | H460 | H1792 | HCC-44 | H1355 | IC₅₀ * | IMR90 |
|---|---|---|---|---|---|---|---|---|---|---|---|---|---|
| 1 | 75 ± | 47 ± | 47 ± | 37 ± | 52 | N.D. | >100 | 52 ± | 60 ± | 45 ± | 41 ± | 62 | N.D. |
| | 0.89 | 1.1 | 0.4 | 1.3 | | | | 1.2 | 1.4 | 0.25 | 0.33 | | |
| 2 | 53 ± | 45 ± | 17 ± | 6.1 ± | 30 | N.D. | 56 ± | 65 ± | 24 ± | 26 ± | 34 ± | 41 | N.D. |
| | 2.1 | 1.7 | 0.8 | 0.76 | | | 1.6 | 2.2 | 0.98 | 1.3 | 1.4 | | |
| 3 | 15 ± | 27 ± | 11 ± | 11 ± | 16 | N.D. | 91 ± | 68 ± | 50 ± | 10 ± | 9.1 ± | 46 | N.D. |
| | 1.1 | 1.2 | 0.63 | 0.96 | | | 1.03 | 0.10 | 1.2 | 1.1 | 1.03 | | |
| 4 | 5.3 ± | 7 ± | 11 ± | 12 ± | 9 | N.D. | 19 ± | 11 ± | 16 ± | 14 ± | 8.1 ± | 14 | N.D. |
| | 0.88 | 0.81 | 0.88 | 1.03 | | | 1.0 | 1.2 | 0.94 | 1.1 | 0.66 | | |
| 5 | 52 ± | 42 ± | 49 ± | 70 ± | 53 | N.D. | 49 ± | >100 | >100 | 32 ± | 12 ± | 59 | N.D. |
| | 0.37 | 1.7 | 0.11 | 1.6 | | | 0.33 | | | 1.6 | 0.72 | | |
| 6 | 55 ± | 26 ± | 62 ± | 5.6 ± | 37 | N.D. | 13 ± | 37 ± | 15 ± | 19 ± | 59 ± | 29 | N.D. |
| | 1.9 | 1.5 | 2.4 | 0.76 | | | 1.03 | 1.8 | 1.1 | 1.4 | 1.7 | | |
| 7 | 6.5 ± | 0.95 ± | 6.3 ± | 1.8 ± | 4 | 8.5 ± | 16 ± | 6.6 ± | 12 ± | 9.2 ± | 7.8 ± | 10 | 63 ± |
| | 0.97 | 1.0 | 0.98 | 0.96 | | 1.2 | 1.3 | 1.02 | 1.04 | 1.02 | 0.61 | | 2.5 |
| 8 | 3.3 ± | 1.1 ± | 5.9 ± | 1.8± | 3 | 17 ± | 9.1 ± | 13 ± | 15 ± | 7.3 ± | 6.4 ± | 11 | 87 ± |
| | 0.93 | 1.1 | 0.97 | 1.05 | | 1.2 | 1.2 | 1.4 | 1.1 | 1.05 | 0.65 | | 4.9 |

* Averaged values.

Notably, compounds 7 and 8 exhibit low micromolar IC50 values across multiple NSCLC cell models, often in the single-digit micromolar range (as low as ˜1 μM in certain cell lines). For example, in an MTT viability assay after 48 h treatment, compound 7 showed IC50 values of ˜0.95 μM in H661 cells and ˜1.8 μM in H1993 cells (both KRAS wild-type), and compound 8 showed IC50 ˜1.1 μM in H661 and ˜1.8 μM in H1993. Both compounds were effective against KRAS-mutant lines as well (e.g., IC50 ˜9 μM in A549 for compound 8). These mixed-ligand Au(I) complexes outperformed simpler Au complexes; for instance, a representative gold(III) complex (compound 1 or 2) had average IC50 on the order of tens of micromolar, and a simpler Au(I) NHC—Br complex (compound 4) had IC50 around 8-16 μM in similar assays. The superiority of the Au-carbene-phosphine compounds is clear and is attributable to their structural features, including increased lipophilicity and stability imparted by the phosphine ligand.

Compounds 7 and 8 showed the greatest potency. For example, compound 7 had IC50 values of 0.95±1.0 μM in H661, 1.8±0.96 μM in H1993, 6.5±0.97 μM in H522, and 7.8±0.61 μM in H1355; the average IC50 across KRAS wild-type lines was ˜4 μM and across KRAS mutant lines ˜10 μM. Compound 8 had IC50 values of 1.1±1.1 μM in H661, 1.8±1.05 μM in H1993, 3.3±0.93 μM in H522, and 6.4±0.65 μM in H1355; averages ˜3 μM (wild-type) and ˜11 μM (mutant). In contrast, a standard chemotherapy drug (cisplatin) typically has IC50 in the 5-20 μM range in similar assays, indicating these gold compounds are comparably potent, if not more so in certain cell lines.

The curves demonstrate that compounds 7 (FIG. 2G) and 8 (FIG. 2H) produce the greatest cytotoxic effects, achieving complete growth inhibition at sub-micromolar concentrations in multiple cell lines. By contrast, gold (III) compounds (e.g., FIG. 2A for compound 1) require much higher concentrations for a similar effect, indicating lower potency, but they still demonstrated a an effect on the lung cancer cells. Normal lung fibroblasts (IMR-90) show significantly less sensitivity.

Gold (III) compounds were less active: compound 1 had IC50 around 30-50 μM in most lines; compound 2 was slightly better (˜6-17 μM in some lines but >50 μM in others). Compounds 5 and 6 (AuBr₃ complexes) had IC50 in the ˜25-60 μM range in various cells, with some cell-specific differences but overall inferior to gold(I) phosphine compounds.

Notably, compound 4 (Au(NHC_BzMe)Br) was relatively potent (IC50 ˜5-16 μM across lines, especially effective in KRAS wild-type H522 ˜5.3 μM), indicating that even without phosphine, a neutral Au(I)-Br complex can have substantial activity, although it lacked the broad efficacy of 7 and 8. Compound 3, the bis-NHC cationic complex, showed mixed results (some lines ˜10-15 μM, others much higher, possibly due to uptake issues for the charged species).

A selectivity assay was performed using normal lung fibroblasts (EVIR-90) for the most promising compounds. Compound 7 had IC50 ˜63±2.5 μM in IMR-90, and compound 8 had IC50˜87±4.9 μM in IMR-90. These values are ˜10-fold higher than the IC50s in cancer cells, suggesting these compounds are significantly less toxic to normal cells. This therapeutic index is encouraging for potential in vivo applications.

### Example 2: TrxR Inhibition

In parallel, enzymatic assays were conducted.

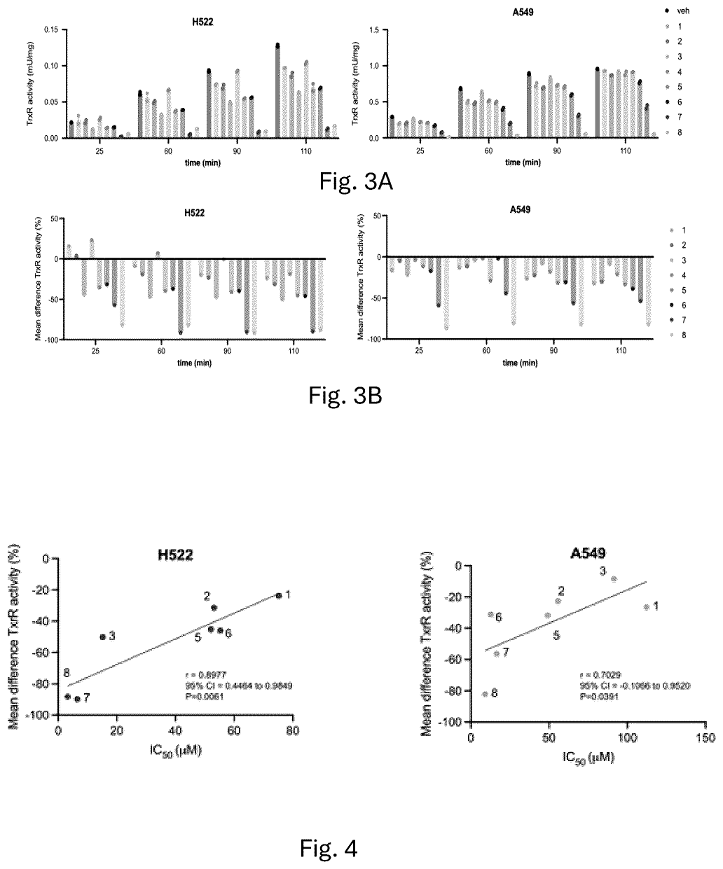

H522 (KRAS wild) and A549 (KRAS mutant) cells were treated with 5 μM of each compound for 24 h. Cell lysates were assayed for TrxR activity. Compounds 7 and 8 showed the largest reduction in TrxR activity, with compound 8 reducing activity by >80% in both cell lines and compound 7 by ˜60-70% (approximate, derived from FIG. 3B). Gold(III) compounds (1,2) reduced TrxR by only ˜20-30%, and tribromides (5,6) by ˜30-40%. There was a strong correlation (Pearson r≈−0.89 in H522) between a compound's TrxR inhibition and its cytotoxic potency. This confirms TrxR as a key pharmacological target of these compounds.

The data, depicted in FIGS. 3A-3B and summarized in Table 3 below, demonstrate a clear reduction in TrxR activity in cells exposed to the gold complexes, with compounds 7 and 8 causing the most pronounced inhibition. In particular, compound 8 reduced TrxR activity to below 20% of control (over 80% inhibition) in both H522 and A549 cells, whereas other compounds showed moderate (compound 5 and 6: ˜30-40% inhibition) to strong (compound 7: ˜60-70% inhibition) effects. This figure supports the conclusion that TrxR is a primary molecular target of the disclosed gold complexes in lung cancer cells, and that the degree of TrxR inhibition correlates with cytotoxic efficacy.

TABLE 3

| | H522 | | | | A549 | | | |
|---|---|---|---|---|---|---|---|---|
| | | 95.00% | | | | 95.00% | | |
| Dunnett's | Mean | CI of | | Adj p | Mean | CI of | | Adj p |
| Comparison | Diff. | Diff. | Summary | Value | Diff. | Diff. | Summary | Value |

25 min

| | | | | | | | | |
|---|---|---|---|---|---|---|---|---|
| 1 vs. veh | 15.84 | −6.504 | ns | 0.2655 | −16.73 | −32.31 | * | 0.0306 |
| | | to 38.19 | | | | to −1.152 | | |
| 2 vs. veh | 3.864 | −18.48 | ns | 0.9981 | −5.148 | −20.73 | ns | 0.918 |
| | | to 26.21 | | | | to 10.43 | | |
| 3 vs. veh | −44.05 | −66.40 | **** | <0.0001 | −21.88 | −37.46 | ** | 0.0027 |
| | | to −21.70 | | | | to −6.300 | | |
| 4 vs. veh | 23.18 | 0.8373 | * | 0.0391 | −3.861 | −19.44 | ns | 0.9819 |
| | | to 45.53 | | | | to 11.72 | | |
| 5 vs. veh | −35.55 | −57.90 | *** | 0.0006 | −11.58 | −27.16 | ns | 0.2233 |
| | | to −13.20 | | | | to 3.996 | | |
| 6 vs. veh | −31.68 | −54.03 | ** | 0.0024 | −17.37 | −32.95 | * | 0.023 |
| | | to −9.338 | | | | to −1.795 | | |
| 7 vs. veh | −57.19 | −79.53 | **** | <0.0001 | −59.2 | −74.78 | **** | <0.0001 |
| | | to −34.84 | | | | to −43.62 | | |
| 8 vs. veh | −81.92 | −104.3 | **** | <0.0001 | −86.87 | −102.5 | **** | <0.0001 |
| | | to −59.57 | | | | to −71.29 | | |

60 min

| | | | | | | | | |
|---|---|---|---|---|---|---|---|---|
| 1 vs. veh | −8.894 | −31.24 | ns | 0.8206 | −13.1 | −28.68 | ns | 0.1319 |
| | | to 13.45 | | | | to 2.479 | | |
| 2 vs. veh | −19.16 | −41.50 | ns | 0.1201 | −11.64 | −27.22 | ns | 0.2189 |
| | | to 3.190 | | | | to 3.934 | | |
| 3 vs. veh | −47.07 | −69.42 | **** | <0.0001 | −4.221 | −19.80 | ns | 0.97 |
| | | to −24.72 | | | | to 11.36 | | |
| 4 vs. veh | 7.116 | −15.23 | ns | 0.9312 | −2.183 | −17.76 | ns | 0.9996 |
| | | to 29.46 | | | | to 13.40 | | |
| 5 vs. veh | −39.68 | −62.03 | *** | 0.0001 | −29.11 | −44.69 | **** | <0.0001 |
| | | to −17.34 | | | | to −13.53 | | |
| 6 vs. veh | −37.22 | −59.57 | *** | 0.0003 | −2.183 | −17.76 | ns | 0.9996 |
| | | to −14.87 | | | | to 13.40 | | |
| 7 vs. veh | −91.41 | −113.8 | **** | <0.0001 | −44.4 | −59.97 | **** | <0.0001 |
| | | to −69.06 | | | | to 28.82 | | |
| 8 vs. veh | −82.1 | −104.4 | **** | <0.0001 | −80.79 | −96.36 | **** | <0.0001 |
| | | to −59.75 | | | | to −65.21 | | |

90 min

| | | | | | | | | |
|---|---|---|---|---|---|---|---|---|
| 1 vs. veh | −20.1 | −42.44 | ns | 0.0938 | −26.55 | −42.13 | *** | 0.0002 |
| | | to 2.252 | | | | to −10.98 | | |
| 2 vs. veh | −23.38 | −45.73 | * | 0.0368 | −22.67 | −38.25 | ** | 0.0018 |
| | | to −1.037 | | | | to −7.090 | | |
| 3 vs. veh | −47.31 | −69.66 | **** | <0.0001 | −8.42 | −24.00 | ns | 0.5454 |
| | | to −24.97 | | | | to 7.159 | | |
| 4 vs. veh | −0.3654 | −22.71 | ns | >0.9999 | −18.13 | −33.71 | * | 0.0163 |
| | | to 21.98 | | | | to −2.556 | | |
| 5 vs. veh | −41.1 | −63.45 | **** | <0.0001 | −31.74 | −47.31 | **** | <0.0001 |
| | | to −18.76 | | | | to −16.16 | | |
| 6 vs. veh | −39.82 | −62.17 | *** | 0.0001 | −31.09 | −46.67 | **** | <0.0001 |
| | | to −17.48 | | | | to −15.51 | | |
| 7 vs. veh | −90.61 | −113.0 | **** | <0.0001 | −56.35 | −71.93 | **** | <0.0001 |
| | | to −68.26 | | | | to −40.77 | | |
| 8 vs. veh | −91.89 | −114.2 | **** | <0.0001 | −82.25 | −97.83 | **** | <0.0001 |
| | | to −69.54 | | | | to −66.68 | | |

110 min

| | | | | | | | | |
|---|---|---|---|---|---|---|---|---|
| 1 vs. veh | −23.89 | −46.24 | * | 0.0316 | −32.38 | −47.96 | **** | <0.0001 |
| | | to −1.545 | | | | to −16.80 | | |
| 2 vs. veh | −31.46 | −53.80 | ** | 0.0026 | −30.44 | −46.02 | **** | <0.0001 |
| | | to −9.111 | | | | to −14.86 | | |
| 3 vs. veh | −50.17 | −72.52 | **** | <0.0001 | −9.068 | −24.65 | ns | 0.4662 |
| | | to −27.83 | | | | to 6.512 | | |
| 4 vs. veh | −18.72 | −41.06 | ns | 0.1345 | −21.37 | −36.95 | ** | 0.0034 |
| | | to 3.631 | | | | to −5.794 | | |
| 5 vs. veh | −45.26 | −67.61 | **** | <0.0001 | −33.68 | −49.26 | **** | <0.0001 |
| | | to −22.91 | | | | to −18.10 | | |
| 6 vs. veh | −46.06 | −68.40 | **** | <0.0001 | −38.86 | −54.44 | **** | <0.0001 |
| | | to −23.71 | | | | to −23.28 | | |
| 7 vs. veh | −89.86 | −112.2 | **** | <0.0001 | −53.76 | −69.34 | **** | <0.0001 |
| | | to −67.51 | | | | to −38.18 | | |
| 8 vs. veh | −88.27 | −110.6 | **** | <0.0001 | −82.25 | −97.83 | **** | <0.0001 |
| | | to −65.92 | | | | to −66.68 | | |

FIG. 4 depicts the correlation between TrxR inhibition and cytotoxic potency of the gold complexes provides a correlation analysis, plotting the mean % TrxR activity (after treatment) against the IC50 of each compound in two cell lines (H522 and A549). The plots illustrate a strong inverse correlation: compounds that are more potent (lower IC50) tend to induce greater TrxR inhibition (lower residual activity). For example, points corresponding to compound 7 and 8 lie in the low-IC50, high-inhibition quadrant, whereas less potent compounds (e.g., 1, 2) cluster at higher IC50 and show only modest TrxR inhibition. The Pearson correlation coefficients are significant, reinforcing that TrxR inhibition is linked to the mechanism of cytotoxic action for these gold compounds.

### Example 3: DHFR Inhibition

Recombinant human DIFR enzyme was incubated with compounds 7 and 8 at varying concentrations, and NADPH consumption was measured to determine enzyme activity. As detailed earlier, compound 8 exhibited allosteric inhibition with an IC50˜15 μM and achieved up to ˜73% inhibition of DHFR at saturating substrate. Compound 7 had a more modest effect (˜50% max inhibition) with IC50 values roughly 11 μM (low [H₂F]) to 24 μM (high [H₂F]).

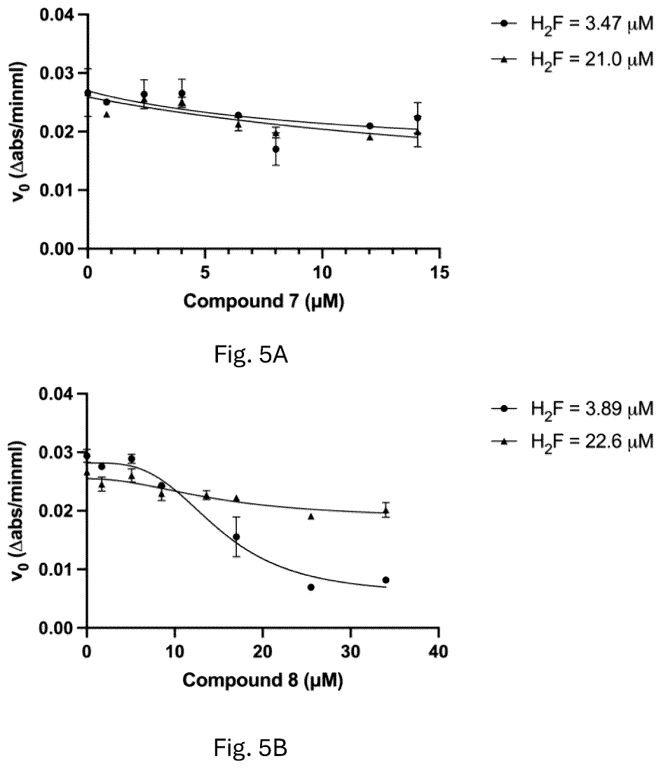

As shown in FIGS. 5A and 5B, compound 8 uniquely functions as a dual inhibitor (TrxR & DHFR), whereas compound 7 is primarily a TrxR inhibitor with ancillary DHFR activity.

In summary, the detailed description above provides a thorough understanding of the structure, preparation, and function of the gold complexes of this invention. These complexes are characterized by their unique coordination environments (especially the NHC—Au—PPh₃ arrangement), their high cytotoxicity toward lung cancer cells, and their dual inhibition of TrxR and DHFR which underpins their therapeutic potential. This dual-target mechanism is expected to produce synergistic anticancer effects, suppressing tumor growth and overcoming resistance pathways. In summary, the invention provides: (a) novel compositions of matter (gold(I/III) complexes with NHC/imidazolyl and phosphine/halide ligands) with demonstrated anticancer efficacy; (b) synthetic methods for preparing these complexes; (c) pharmaceutical formulations comprising the complexes; and (d) methods of treating lung cancer (and potentially other malignancies) using these complexes. By integrating potent TrxR inhibition with an antifolate (DHFR inhibitory) effect, the compounds of the invention address the need for multi-faceted cancer therapeutics to overcome drug resistance and improve treatment outcomes in NSCLC.

### Example 4: Materials and Methods

All the reactants needed for the preparation of compounds 1-8 were purchased from Merck and used without any further purification. Solvents were bought from Carlo Erba (Milano, Italy) and freshly distilled before use. Celite and molecular sieves were bought from Supelco (Merck). The TrxR assay Kit (ab83463) and the Proteinase Inhibitor Cocktail (ab65621) were purchased from Abcam (Waltham, MA, USA). The plasmid pET17b encoding the cDNA for the hDHFR was kindly gifted by prof. C. Robert Matthews (University of Massachusetts Medical School); Hepes buffer, K₂HPO₄, NADPH, dihydrofolic acid, and β-mercaptoethanol were purchased from Merck Life Science (Milan, Italy). BL21 competent E. coli cells were purchased from Novagen®, Merck KGaA (Darmstadt, Germany)

Elemental analyses (C, H, N, S) were performed in-house with a Fisons Instruments 1108 CHNS-O Elemental Analyser. Melting points were taken on an SMP3 Stuart Scientific Instrument. IR spectra were recorded from 4000 to 600 cm⁻¹ with a Perkin-Elmer SPECTRUM ONE System FT-IR instrument. IR annotations used: br=broad, m=medium, s=strong, sh=shoulder, vs=very strong, w=weak, and vw=very weak. ¹H NMR spectra were recorded on an Oxford-400 Varian spectrometer (400.4 MHz for ¹H). Chemical shifts, in ppm, for ¹H NMR spectra are relative to internal Me4Si. NMR annotations used: br=broad, d=doublet, dd=double doublet, t=triplet, m=multiplet, s=singlet. UV-Vis spectra were acquired using the Shimadzu UV-2700i spectrophotometer, equipped with the Shimadzu CPS-100 Peltier, at 298 K (Shimadzu, Kyoto, Japan). Electrospray mass spectra (ESI-MS) were obtained in positive- or negative-ion mode on a high-performance liquid chromatography (HPLC) Alliance 2695 Waters coupled with a single quadrupole mass spectrometry (Waters Micromass ZQ, Milford, MA, USA). The mobile phase was acetonitrile or methanol, and the compounds were dissolved in the mobile phase with an approximate concentration of 0.1 mM. The injection volume was 1 μL, and the flow rate was 200 μL min⁻¹. Nitrogen was employed both as a drying and nebulizing gas. Capillary voltages were typically 4000 V and 3500 V for the positive- and negative-ion modes, respectively. Confirmation of all major species in this ESI-MS study was aided by a comparison of the observed and predicted isotope distribution patterns, the latter calculated using ChemDraw 20.1 computer program.

### Cell Lines

NSCLC cell lines were obtained from the Hamon Center for Therapeutic Oncology Research (UT Southwestern Medical Center). IMR-90 human lung fibroblasts were from ATCC (CC-186). All cell lines were maintained and treated in RPMI medium (Cytiva (Shanghai, China), #SH30027), supplemented with 10% heat-inactivated FBS (Millipore Sigma (Darmstadt, Germany), F0394)

### MTT Assays

The enzymatic activity of TrxR in the A549 and H522 cell lysates was measured using the TrxR assay Kit (Abcam ab83463) following the manufacturer's instructions. This kit is based on the TxrR-mediated reduction of the colorless DTNB into TNB2—(yellow). Briefly, cells were plated to reach 70% confluence on a 100-mm dish in one day. They were treated with 5 μM of the gold compounds or the equivalent quantity of DMSO for 24 h. Then, cells were washed in PBS 1×, detached, and lysed in 100 μL of cold TrxR assay buffer supplemented with the Proteinase Inhibitor Cocktail. 20 μg and 40 μg of total proteins were used for the A549 and the H522, respectively. The DTNB substrate was added to samples with or without TrxR inhibitor (provided with the kit) in 96-well plates. The OD412 was measured soon after the substrate addition (Al) and then after 25, 60, 90, and 110 min (An) at 25° C. using a FLUOstar Omega Microplate Reader (BGM Labtech, Chicago, IL, USA). Data analysis was performed following the manufacturer's instructions. The OD of TNB2, generated by TrxR in every condition, is: ΔA_{412 nm}=(A_{nAB}−A_{nINH}) ˜(A_{B}−A_{1INH}) where ΔB is the assay buffer, and INH is the inhibitor.

The TrxR activity is: ΔB(Tn−T1)×mg of protein lysate×sample dilution factor=(nmol/min)/mg=mU/mg where ΔB is the TNB amount calculated applying ΔA_{412 nm} to TNB standard curve (in nmol), T1 is the time of the first reading (A_{1AB}, and A_{1INH}) (in min), Tn is the time of the second reading (A_{nAb} and A_{nNH}) (in min). The residual TxrR activity (%) was calculated by normalizing the TxrR activity for each compound with the matching vehicle values at every n time point.

Cell-Free Human Dihydrofolate Reductase (hDHFR) Enzymatic Activity

Recombinant human DHFR was overexpressed and purified from pET17b (+) encoding the hDHFR gene/BL21 (Novagene® (Hong Kong, China)) E. coli cells as described in the literature. The protein purity was verified as a single band in Coomassie-stained SDS-PAGE. The concentration of the hDHFR was measured spectrophotometrically at 280 nm using the extinction coefficient of 28,400 M−1 cm−1 and resulted to be equal to 14 μM. The activity of the DHFR was followed by recording the absorbance over time at 340 nm in a UV-VIS spectrophotometer Shimadzu UV-2450. The procedure consisted of mixing the 50 mM Hepes buffer (pH 7.3) with saturating concentration NADPH (85-90 mM) and 0.07 mM hDHFR, incubating at 37° C. for 5 min, and starting the reaction by adding H2F at various concentrations. When testing the inhibition capacity of gold complexes, the reaction mixture was prepared by adding increasing concentrations of the tested compounds before the incubation at 37° C. Since the gold complexes were solubilized in 100% methanol (HPLC-grade, Carlo Erba), the enzymatic activity of hDHFR was also assessed in the presence of the equal volumes of MeOH used in the assays with the gold complexes and eventually subtracted. Data are represented as mean±SEM of 2 independent experiments and analyzed according to best-fit models using GraphPad Prism (v10).

Aspects of the present disclosure can be described with reference to the following numbered clauses, with preferred features laid out in dependent clauses.

1. In one aspect, alone or in combination with any other aspect disclosed herein, the present disclosure relates to a method of treating lung cancer in a subject in need thereof, the method comprising administering to the subject an effective amount of an imidazolate gold compound according to Formula I, or a pharmaceutically acceptable salt, racemate, or enantiomer thereof, wherein Formula I is a gold complex having a structure Au(L)(L′)ₙ, wherein n is an integer from 1 to 3; L is an imidazolyl-based N-heterocyclic carbene (NHC) ligand; and each L′ is independently selected from an imidazolyl-based NHC ligand, a triaryl phosphine, or a halide.

2. In one aspect, alone or in combination with any other aspect disclosed herein, the present disclosure relates to a method of treating lung cancer in a subject in need thereof, wherein the lung cancer is KRAS-wild type lung cancer.

3. In one aspect, alone or in combination with any other aspect disclosed herein, the present disclosure relates to a method of treating lung cancer in a subject in need thereof, wherein the imidazolate gold compound is selected from the group consisting of 1,3-dimethylimidazol-2-ylidene)gold(III) trichloride (compound 1), (1-benzyl-3-methylimidazol-2-ylidene)gold(III) trichloride (compound 2), bis(1,3-dimethylimidazol-2-ylidene)gold(I) complex with a bromidoaurate counterion ([Au(NHC)₂]⁺[AuBr₂]⁻) (compound 3), (1-benzyl-3-methylimidazol-2-ylidene) gold (I) bromide (compound 4), (1,3-dimethylimidazol-2-ylidene)gold(III) tribromide (compound 5), (1-benzyl-3-methylimidazol-2-ylidene)gold(III) tribromide (compound 6), 1-benzyl-4,5-dichloroimidazolyl gold (I) triphenylphosphine (compound 7) or (1,3-dimethylimidazol-2-ylidene) triphenylphosphine, gold (I) chloride (compound 8, and combinations thereof.

4. In one aspect, alone or in combination with any other aspect disclosed herein, the present disclosure relates to a method of treating lung cancer in a subject in need thereof, wherein the NHC ligand is a substituted imidazol-2-ylidene of the form:

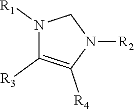

wherein R¹ and R² are substituents on the ring nitrogens selected from C₁-C₆ alkyl or benzyl, and R³ and R⁴ are substituents at the 4- and 5-positions of the imidazole ring selected from H, halogen, or C₁-C₂ alkyl

5. In one aspect, alone or in combination with any other aspect disclosed herein, the present disclosure relates to a method of treating lung cancer in a subject in need thereof, wherein L is selected from the group consisting of 1,3-dimethylimidazol-2-ylidene, 1-benzyl-3-methylimidazol-2-yliden, and 1-benzyl-4,5-dichloroimidazol-2-ide.

6. In one aspect, alone or in combination with any other aspect disclosed herein, the present disclosure relates to a method of treating lung cancer in a subject in need thereof, wherein L is 1-benzyl-3-methylimidazol-2-yliden.

7. In one aspect, alone or in combination with any other aspect disclosed herein, the present disclosure relates to a method of treating lung cancer in a subject in need thereof, wherein L′ is a triaryl phosphine, or a halide.

8. In one aspect, alone or in combination with any other aspect disclosed herein, the present disclosure relates to a method of treating lung cancer in a subject in need thereof, wherein L′ is a triphenyl phosphine.

9. In one aspect, alone or in combination with any other aspect disclosed herein, the present disclosure relates to a method of treating lung cancer in a subject in need thereof, wherein the imidazolate gold compound is administered by injection or infusion.

10. In one aspect, alone or in combination with any other aspect disclosed herein, the present disclosure relates to a method of treating lung cancer in a subject in need thereof, further comprising administering to the subject one or more additional anti-cancer agents.

11. In one aspect, alone or in combination with any other aspect disclosed herein, the present disclosure relates to a method of treating lung cancer in a subject in need thereof, wherein the one or more additional anti-cancer agents is selected from the group consisting of a chemotherapeutic agent, an immunotherapeutic agent, and radiation therapy.

12. In one aspect, alone or in combination with any other aspect disclosed herein, the present disclosure relates to a method of treating lung cancer in a subject in need thereof, wherein the chemotherapeutic agent is selected from the group consisting of cisplatin, carboplatin, paclitaxel, albumin-bound paclitaxel, docetaxel, gemcitabine, vinorelbine, etopside, pemetrexed, and combinations thereof.

13. In one aspect, alone or in combination with any other aspect disclosed herein, the present disclosure relates to a method of treating lung cancer in a subject in need thereof, wherein the immunotherapeutic agent is selected from the group consisting of nivolumab, pembrolizumab, cemiplimab, atezolizumab, durvalumab, ipilimumab, tremelimumab, and combinations thereof.

14. In one aspect, alone or in combination with any other aspect disclosed herein, the present disclosure relates to a method of treating lung cancer in a subject in need thereof, wherein the subject is a human.

15. In one aspect, alone or in combination with any other aspect disclosed herein, the present disclosure relates to a method of treating lung cancer in a subject in need thereof, wherein the imidazolate gold compound has a formula selected from the group consisting of Au(L)X₃; Au(L)(PPh₃); Au(L)X; or Au(L)(PPh₃)X; Au(L)₂]⁺A⁻, wherein X is a halide, PPh₃ is triphenyl phosphine, and A⁻ is a halide or an AuX₂⁻.

16. In one aspect, alone or in combination with any other aspect disclosed herein, the present disclosure relates to a method of treating lung cancer in a subject in need thereof, wherein the imidazolate gold compound is

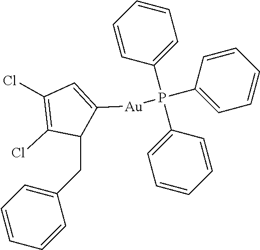

17. In one aspect, alone or in combination with any other aspect disclosed herein, the present disclosure relates to a method of treating lung cancer in a subject in need thereof, wherein the imidazolate gold compound is

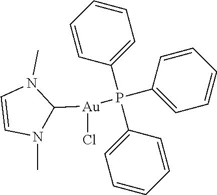

18. In one aspect, alone or in combination with any other aspect disclosed herein, the present disclosure relates to a method of treating lung cancer in a subject in need thereof, wherein administering the imidazolate gold compound inhibits TrxR and/or DHFR.

19. In one aspect, alone or in combination with any other aspect disclosed herein, the present disclosure relates to a method of treating lung cancer in a subject in need thereof, wherein inhibition of TrxR and/or DHFR results in at least a 20% reduction in activity

20. In one aspect, alone or in combination with any other aspect disclosed herein, the present disclosure relates to a compound or a pharmaceutically acceptable salt, racemate, or enantiomer thereof, having the formula of compound 8:

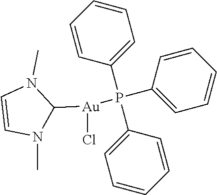

All documents cited are incorporated herein by reference; the citation of any document is not to be construed as an admission that it is prior art with respect to the present invention.

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention

1. A method of treating lung cancer in a subject in need thereof, the method comprising administering to the subject an effective amount of an imidazolate gold compound according to Formula I, or a pharmaceutically acceptable salt, racemate, or enantiomer thereof, wherein Formula I is a gold complex having a structure Au(L)(L′)ₙ, wherein:
n is an integer from 1 to 3;
L is an imidazolyl-based N-heterocyclic carbene (NHC) ligand; and
each L′ is independently selected from an imidazolyl-based NHC ligand, a triaryl phosphine, or a halide.

2. The method according to claim 1, wherein the lung cancer is KRAS-wild type lung cancer.

3. The method according to claim 1, wherein the imidazolate gold compound is selected from the group consisting of 1,3-dimethylimidazol-2-ylidene)gold(III) trichloride (compound 1), (1-benzyl-3-methylimidazol-2-ylidene)gold(III) trichloride (compound 2), bis(1,3-dimethylimidazol-2-ylidene)gold(I) complex with a bromidoaurate counterion ([Au(NHC)₂]⁺[AuBr₂]⁻) (compound 3), (1-benzyl-3-methylimidazol-2-ylidene) gold (I) bromide (compound 4), (1,3-dimethylimidazol-2-ylidene)gold(III) tribromide (compound 5), (1-benzyl-3-methylimidazol-2-ylidene)gold(III) tribromide (compound 6), 1-benzyl-4,5-dichloroimidazolyl gold (I) triphenylphosphine (compound 7) or (1,3-dimethylimidazol-2-ylidene) triphenylphosphine, gold (I) chloride (compound 8, and combinations thereof.

4. The method according to claim 1, wherein the NHC ligand is a substituted imidazol-2-ylidene of the form: 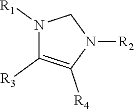
wherein R¹ and R² are substituents on the ring nitrogens selected from C₁-C₆ alkyl or benzyl, and R³ and R⁴ are substituents at the 4- and 5-positions of the imidazole ring selected from H, halogen, or C₁-C₂ alkyl.

5. The method of claim 1, wherein L is selected from the group consisting of 1,3-dimethylimidazol-2-ylidene, 1-benzyl-3-methylimidazol-2-yliden, and 1-benzyl-4,5-dichloroimidazol-2-ide.

6. The method of claim 1, wherein L is 1-benzyl-3-methylimidazol-2-yliden.

7. The method of claim 1, wherein L′ is a triaryl phosphine, or a halide.

8. The method of claim 1, wherein L′ is a triphenyl phosphine.

9. The method of claim 1, wherein the imidazolate gold compound is administered by injection or infusion.

10. The method of claim 1, further comprising administering to the subject one or more additional anti-cancer agents.

11. The method of claim 10, wherein the one or more additional anti-cancer agents is selected from the group consisting of a chemotherapeutic agent, an immunotherapeutic agent, and radiation therapy.

12. The method of claim 11, wherein the chemotherapeutic agent is selected from the group consisting of cisplatin, carboplatin, paclitaxel, albumin-bound paclitaxel, docetaxel, gemcitabine, vinorelbine, etopside, pemetrexed, and combinations thereof.

13. The method of claim 11, wherein the immunotherapeutic agent is selected from the group consisting of nivolumab, pembrolizumab, cemiplimab, atezolizumab, durvalumab, ipilimumab, tremelimumab, and combinations thereof.

14. The method of claim 1, wherein the subject is a human.

15. The method of claim 1, wherein the imidazolate gold compound has a formula selected from the group consisting of Au(L)X₃; Au(L)(PPh₃); Au(L)X; or Au(L)(PPh₃)X; Au(L)₂]⁺A⁻, wherein X is a halide, PPh₃ is triphenyl phosphine, and A⁻ is a halide or an AuX₂⁻.

16. The method of claim 1, wherein the imidazolate gold compound is

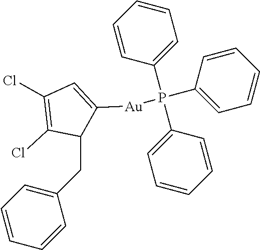

17. The method of claim 1, wherein the imidazolate gold compound is

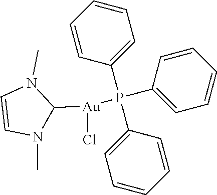

18. The method of claim 1, wherein administering the imidazolate gold compound inhibits TrxR and/or DHFR.

19. The method of claim 18, wherein inhibition of TrxR and/or DHFR results in at least a 20% reduction in activity.

20. A compound or a pharmaceutically acceptable salt, racemate, or enantiomer thereof, having the formula of compound 8: [chemical structure]
